(19) United States
(12) Patent Application Publication
(10) Pub. No.: US 2022/0151818 A1
(43) Pub. Date: May 19, 2022

(54) EXTERNAL HEAD APPARATUS FOR REDUCING THE TONE OF MASTICATORY MUSCLES IN PATIENTS WITH BRUXISM AND/OR OTHER CRANIOMANDIBULAR DYSFUNCTIONS (CMD)

(71) Applicant: RELAXBOGEN GMBH, Hameln (DE)

(72) Inventors: Christoph SEIDENSTÜCKER, Hameln (DE); Amir Moazzen SAFAEI, Berlin (DE)

(73) Assignee: RELAXBOGEN GMBH, Hameln (DE)

(21) Appl. No.: 17/433,599

(22) PCT Filed: Mar. 23, 2020
(86) PCT No.: PCT/EP2020/057948

§ 371 Date: Aug. 25, 2021

(30) Foreign Application Priority Data
Mar. 27, 2019 (DE) 10 2019 002 200.9
Mar. 27, 2019 (DE) 20 2019 001 429.2

(51) Int. Cl. A61F 5/56; A61H 39/04; A61H 23/00

(52) U.S. Cl. CPC A61F 5/56; A61H 39/04; A61H 23/00; A61H 2201/5058; A61H 2201/0207; A61H 2201/1607; A61F 2005/563

(57) ABSTRACT

An improved external head apparatus for reducing the tone of the masticatory muscles in patients with bruxism and/or other craniomandibular dysfunctions (CMD) includes two curved spring-tempered wires and two separate connecting elements. The external head apparatus has a plane of symmetry, which passes through the center of the curved spring-tempered wires. The two separate connecting elements are situated on different sides of the plane of symmetry.

### BACKGROUND OF THE INVENTION

### 1. Field of the Invention

The present invention relates to an improved external head apparatus for reducing the tone of the masticatory muscles in patients with bruxism and/or other craniomandibular dysfunctions (CMD) as well as to a kit for constructing corresponding external head apparatuses.

### 2. Discussion of the Related Art

CMD patients, especially those who are suffering from bruxism (grinding and/or clenching of the teeth) frequently exhibit pains in the region of the masticatory muscles and of the temporomandibular joints. CMD patients regularly have muscular dysbalances in the jaw region due to increased muscle activity. This muscular hyperactivity intensifies the symptoms of the patients, increases the bruxism and thereby favors faster wear of the teeth. Furthermore, the dysbalances may spread via the muscle chains in the entire supporting and locomotor system and lead to consequential symptoms here.

The standard therapy in this situation is usually to create a bite splint to begin with. Besides protecting the teeth, elimination of movement patterns that may have developed (sliding trajectories of the teeth during grinding movements) is achieved hereby. Since the conventional bite splint, which is inserted in the mouth, usually can be worn only at night, practically no influence on the muscle tone is achieved during the day. Moreover, because of the poor wearing comfort, such a splint often achieves only moderate acceptance by the patients. Therefore a continuing need exists to develop, for the treatment of CMD patients, other approaches that are able to supplement, support or replace the established therapy with the bite splint.

In order to prevent bruxism (grinding and/or clenching of the teeth), various solutions have been proposed in the prior art, such as improved bite splints (e.g. U.S. Pat. Nos. 4,304,227 A; 4,169,473 A; or 5,823,193 A) or special nasal strips (DE 10034969 A1). Many of the solutions disclosed in the prior art (e.g. EP 0654981 B1; U.S. Pat. Nos. 5,586,562 A; 5,553,626 A; 5,490,520 A; 4,989,616 A; 4,976,618 A; 5,190,051; 5,078,153 A or 4,995,404 A) are based, for example, on the principle that an electronic or electromechanical detector is disposed in the mouth between the upper and lower dental arches and is connected to a further apparatus, which in the event of grinding emits an audible or other kind of alarm signal. Other devices measure the extent of teeth grinding by the electrical potential present at the large masticatory muscle (masseter muscle) (e.g. U.S. Pat. Nos. 4,934,378 A; 4,715,367 A; 4,669,477 A; or WO 98/51 218 A1) and measured through the skin, and they trip an alarm signal that depends on this measurement. As a disadvantage in these approaches, it is regularly found that the corresponding devices are instrumentally complex and/or poorly effective and/or can be used only during sleeping phases and/or are sensed to be uncomfortable in terms of wearing comfort.

As alternatives to those, external head apparatuses for reducing the tone of the masticatory muscles in patients with bruxism and/or other craniomandibular dysfunctions (CMD) have been successfully established in recent years, such as are disclosed in documents DE 102010033224 B4 and DE 202011103819 U1, for example. Corresponding external head apparatuses achieve the desired effect by constantly exerting, throughout the wearing duration, gentle pressure on the trigger points and fasciae of the two large masticatory muscles, the masseter muscle and the temporal muscle, respectively on the right and left of the head of the patient, and thereby reduce the tone of the masticatory muscles in patients with bruxism and/or other craniomandibular dysfunctions (CMD). These external head apparatuses avoid the disadvantages described in the foregoing and have now become successfully established on the market, especially due to their usability throughout the entire day and their combinability—considered to be particularly advantageous—with the established bite splint therapy.

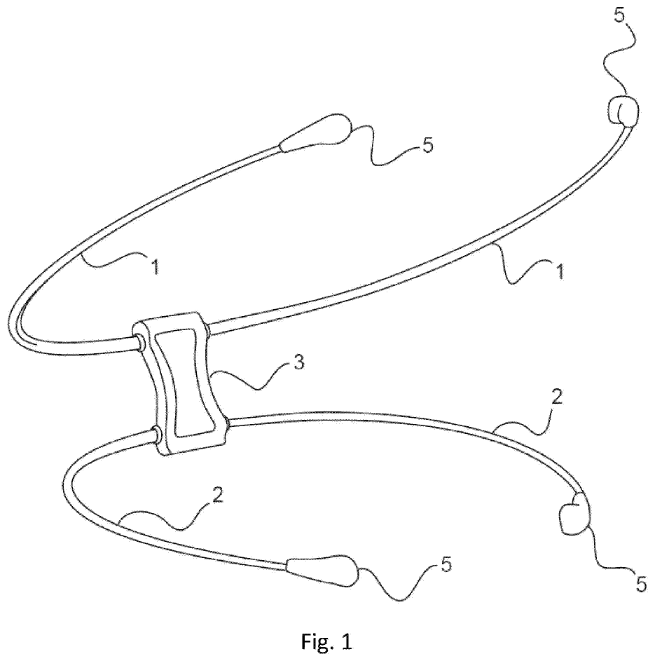

In recent years, corresponding external head apparatuses have undergone steady further development, in order in particular to increase the effectiveness, wearing comfort and durability as well as to lower the production costs. As a result of this continuing further development, the prior art in the field of the corresponding head apparatuses is best represented by the most up-to-date model of corresponding head apparatuses currently available on the market. A corresponding external head apparatus is disclosed, for example, in J. Lambers; Zeitschrift für Kraniomandibuläre Funktion 2018; 10(2):147-159 as well as in J. Lambers et al.; Kieferorthopädie Nachrichten; Special Printed Issue No. 6/15, June, vol. 13, and in J. Lambers et al.; Manuelle Medizin; DOI 10.1007/s00337-016-0169-9. This prior art is also illustrated in FIG. 1. Such a head apparatus comprises two curved spring-tempered wires with bearing surfaces at the ends, which are joined only by one single connecting element.

Despite the fundamentally advantageous properties of corresponding external head apparatuses, a constant need exists to further improve the effectiveness, wearing comfort and durability. In particular, it is considered disadvantageous in some cases that, while the head apparatus is being worn, the position of the single connecting element—necessarily disposed centrally for structural reasons—in the middle of the neck is felt to be uncomfortable, especially in sleeping position, since it is pressed against the neck by the pillow or the mattress. Likewise, it has been found in individual cases, depending on the individual anatomical characteristics of the patients, that the path of the spring-tempered wires underneath and above the ears in corresponding devices is felt to be disadvantageous. Furthermore, the connecting element in the configuration according to the prior art is subjected to relatively high mechanical loads while the head apparatus is being put on and taken off, whereby relatively high-grade and expensive materials must be used as the connecting element in order to assure durability. In addition, it is desirable to further improve not only the seating stability of the head apparatus, especially for use in sleeping phases, but also the effectiveness of tone reduction.

### SUMMARY OF THE INVENTION

The present invention is therefore based on the object of eliminating or at least alleviating the disadvantages described in the foregoing of known external head apparatuses and of achieving the desired improvements. Furthermore, it was a further object of the invention to specify a kit for constructing an external head apparatus, which kit permits individual construction and comfortable adaptation of the external head apparatus to the needs of the patient directly by the treating physician or physiotherapist. Further objects will become evident from the present description.

### BRIEF DESCRIPTION OF THE INVENTION

The foregoing objects are achieved by an external head apparatus and a kit for constructing an external head apparatus as defined in the attached claims. Preferred configurations according to the invention will become apparent from the dependent claims.

In the drawing:

FIG. 1 shows an external head apparatus according to the prior art.

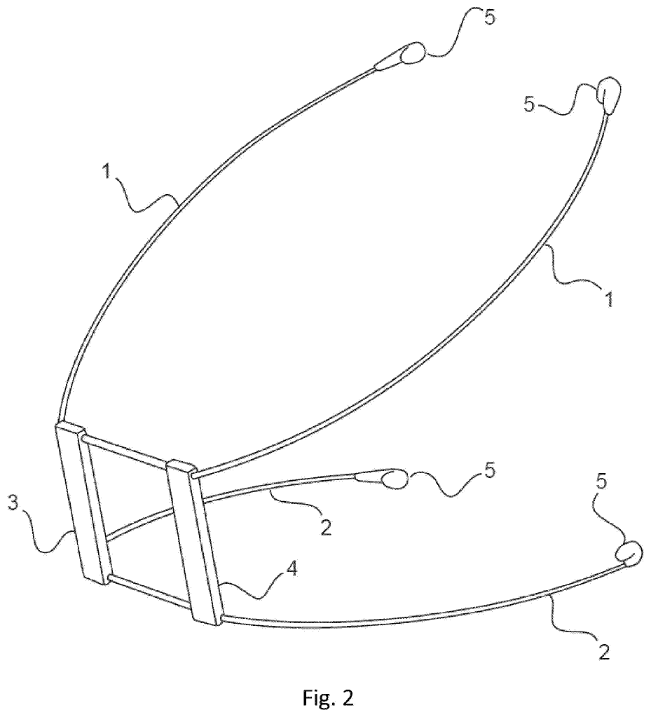

FIG. 2 shows a first view of an external head apparatus according to the invention.

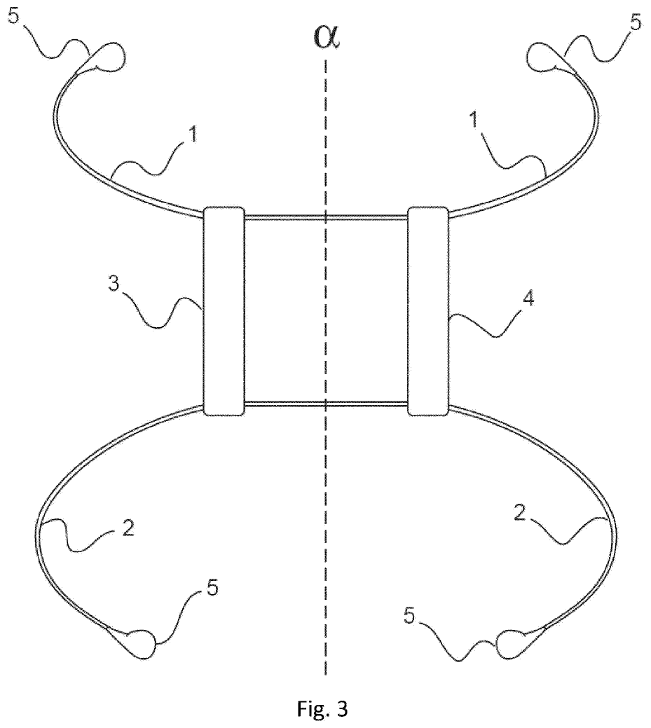

FIG. 3 shows a second view of an external head apparatus according to the invention.

Therein the reference symbols denote the following:

1 a first curved spring-tempered wire,

2 a second curved spring-tempered wire,

3 a first separate connecting element,

4 a second separate connecting element, and

5 supports

### DETAILED DESCRIPTION OF THE INVENTION

The present invention relates in particular to an external head apparatus for reducing the tone of the masticatory muscles in patients with bruxism and/or other craniomandibular dysfunctions (CMD), comprising

(i) two curved spring-tempered wires, and

(ii) two separate connecting elements,

wherein supports are disposed respectively at both ends of the curved spring-tempered wires,

wherein the curved spring-tempered wires are joined to one another by the separate connecting elements in such a way that the external head apparatus can be fixed on a human head,

wherein the curved spring-tempered wires joined to one another are shaped in such a way that, during fixation of the external head apparatus on a human head, pressure due to the supports disposed at the ends of the two or more curved spring-tempered wires is exerted on trigger points and/or fasciae of the two large masticatory muscles, the masseter muscle and the temporal muscle, respectively on the right and left side of the human head,

wherein the external head apparatus has a plane of symmetry that intersects the two curved spring-tempered wires, and

wherein the two separate connecting elements are situated on different sides of the plane of symmetry.

Spring-tempered wires are known to the person skilled in the art. A curved spring-tempered wire is obtained when the spring-tempered wire is not straight but instead has a curvature. A curved spring-tempered wire within the meaning of the present invention may be curved as an open circle or a bow, for example. Although configurations with two curved spring-tempered wires are usually particularly advantageous due to their high wearing comfort, it is also possible to use specific head apparatuses that, by the use of three or more curved spring-tempered wires, advantageously address still further muscle regions or achieve enhanced stabilization by the additional wires. The person skilled in the art therefore understands that the external head apparatus according to the invention comprises no fewer than two curved spring-tempered wires, although an apparatus with in total four curved spring-tempered wires may also be in accordance with the invention.

A separate connecting element within the meaning of the present invention is any element by which two spring-tempered wires can be joined securely to one another, especially in such a way that an entirely free movement of the two spring-tempered wires relative to one another is prevented. Corresponding separate connecting elements are known to the person skilled in the art, consist of commercially common materials and can be machined with established tools. For example, the separate connecting elements may be plastic elements, in which recesses for receiving the spring-tempered wires are drilled with standard tools, so that the spring-tempered wires can be fixed therein. Within the meaning of the present invention, it is to be noted that the connecting elements must be separate, i.e. different from one another and from the spring-tempered wires. For example, if the spring-tempered wires were twisted, adhesively bonded or welded together, this would be just as little a joining by separate connecting elements as a single connecting element, which contacts the spring-tempered wires only in two areas separated from one another. The connecting elements are in contact with the spring-tempered wires respectively via joint areas having length dimensions that may vary. Within the meaning of this invention, what is understood by the spacing between two separate connecting elements relative to a spring-tempered wire is the spacing between the area of the joint, i.e. the area of contact of the first separate connecting element with the spring-tempered wire, and the area of the joint of the second separate connecting element with the spring-tempered wire.

According to the invention, the curved spring-tempered wires respectively have a support at both ends. A support may be, for example, merely a thickening of the spring-tempered wire or a separately disposed structural part, for example of plastic, and it functions for better distribution of the support intensity to the muscle. Preferably, a separate support is disposed respectively at both ends of the curved spring-tempered wires, wherein the support preferably consists of plastic.

According to the invention, the curved spring-tempered wires are joined to one another by two separate connecting elements in such a way that the external head apparatus can be fixed on a human head. In particular, this means that an external head apparatus according to the invention may be attached without additional fastening means to a human head in such a way that it can indeed be put on and taken off with exertion of gentle force, but in everyday use it shifts only slightly and does not slip off or fall off, for example due to a mere movement of the head or during sleep. According to the invention, corresponding fixation is achieved by the arrangement of the two curved spring-tempered wires relative to one another by the two separate connecting elements, so that the curved spring-tempered wires and/or the supports disposed at the ends exert gentle pressure bilaterally on the sides of the head. In addition, further fixation of the head apparatus can also be supported by having the spring-tempered wires rest on the ears. According to the invention, the supports in the as-worn condition, i.e. after fixation on a human head, are situated in the facial region of the person, whereas the spring-tempered wires usually extend in curved manner behind the head.

According to the invention, the spring-tempered wires joined to one another are shaped in such a way that, during fixation of the external head apparatus on a human head, pressure due to the supports disposed at the ends is exerted on trigger points and/or fasciae of the two large masticatory muscles, the masseter muscle and the temporal muscle, respectively on the right and left side of the human head. It is upon this that the fundamental benefit of corresponding external head apparatuses is based, as is described more extensively in the prior art (see DE102010033224 B4, for example).

An external head apparatus according to the invention has a plane of symmetry that intersects the two curved spring-tempered wires. By constructing a mirror image of the one side of the head apparatus relative to this plane, it is possible to illustrate it on the other side of the apparatus. The corresponding plane of symmetry is illustrated in FIG. 3. Within the meaning of this invention, the plane of symmetry preferably extends through the head apparatus in such a way that half of the spring-tempered wires and of the supports disposed at their ends lie respectively on one side and half on the other side of the plane of symmetry. When the head apparatus is fixed on a human head, i.e. is in the as-worn condition, the plane of symmetry of the external head apparatus preferably coincides substantially with the plane of symmetry of the head. Particularly preferably, the plane of symmetry is oriented perpendicular to the plane of symmetry.

It is entirely within the scope of understanding of the person skilled in the art to assume, in connection with the present invention, the existence of a plane of symmetry even if the ideal symmetry is slightly distorted, i.e. a plane of symmetry is assumed according to the invention when the degree of coincidence achieved by constructing a mirror image relative to the plane of symmetry is greater than 95%, preferably greater than 98%, particularly preferably greater than 99%.

Corresponding deviations from ideal symmetry may be determined by the person skilled in the art by simple surveying techniques or by the use of image-processing software, and in practice they may result, for example, from minor fabrication defects or due to individual adaptations to human heads that are not completely symmetric.

Surprisingly, it has been found that the disadvantages known from the prior art and described in the foregoing may be avoided when, instead of one individual connecting element, two separate connecting elements are used, provided they are situated according to the invention on different sides of the plane of symmetry.

In such a configuration according to the invention, none of the connecting elements rests directly on the spinal column of the patient when in the as-worn condition, but instead the separate connecting elements are structurally displaced laterally to the left and right of the spinal column, so that a pillow or a mattress can exert only a slight pressure via the connecting element against the head of the patient. Head apparatuses according to the invention therefore have substantially enhanced wearing comfort and improved end user acceptance, especially during use in the sleeping phase.

In addition, it has proved particularly favorable for the head apparatuses according to the invention that, due to the positioning of two connecting elements, which are shifted away from the spinal column and closer to the ears compared with the prior art, it is particularly easily possible, by choosing suitable connecting elements (especially in terms of size), to increase the wearing comfort even for such patients whose anatomy in the region of the ears has led to fitting problems in configurations according to the prior art. In particular, a gentle slope can be better achieved in the region of the ears with external head apparatuses according to the invention, without diminishing the effect of the external head apparatus on the masticatory muscles.

Surprisingly, it has been shown for external head apparatuses according to the invention that the durability, especially with respect to material failures in the joint areas, is distinctly improved compared with the prior art, even when the total mass of the two separate connecting elements corresponds only to the mass of the single connecting element known from the prior art. Without intending to be bound to a particular theory, this is attributed to the fact that the mechanical load when external head apparatuses according to the invention are being put on and taken off is better distributed to the two connecting elements, wherein especially the lever arm, i.e. the force transmission during bending of the curved spring-tempered wires, is shorter. In practice, it has been shown that it is now possible, due to the configuration of the external head apparatus according to the invention, to fabricate the connecting elements from such plastics that are mechanically much less resistant to load. Thereby greater flexibility is achieved for the use of various plastics, which are preferred with respect to other properties. Besides the use of bio-based plastics, the possibility is also opened up, for example, of constructing external head apparatuses much more inexpensively. Completely contrary to the original expectation, therefore, the construction costs and the material demands are not increased by the configuration of the external head apparatus according to the invention, but instead they can even be reduced in many cases.

In addition, it has been shown in our own tests that the general (subjective) wearing comfort of external head apparatuses according to the invention is rated as higher on average in comparison with the external head apparatuses from the prior art. Advantageously, no negative impacts of the configuration according to the invention on the effectiveness of reducing the tone of the masticatory muscles in patients with bruxism and/or other craniomandibular dysfunctions (CMD) have been observed due to the external head apparatus.

Preference is given to an external head apparatus according to the invention (preferably identified as in the foregoing as being preferred) wherein the curved spring-tempered wires consist of a stainless steel,

and/or

wherein the connecting elements consist of a material that is selected from the group consisting of structural and high-performance plastics, preferably selected from the group consisting of PEEK, PA, POM, PC and PEK.

and/or

wherein the supports consist of a material that is selected from the group consisting of structural and high-performance plastics, preferably selected from the group consisting of PEEK, PA, POM, PC and PEK,

Correspondingly preferred external head apparatuses are particularly advantageous, because the cited materials for construction of the external head apparatus have particularly favorable processing properties and do not adversely influence the total weight of the external head apparatus, and because the corresponding materials have proved particularly resistant and durable in practice for the specific load profile that an external head apparatus according to the invention experiences.

Preference is given to an external head apparatus according to the invention (preferably identified as in the foregoing as being preferred), wherein, for fixation of the external head apparatus on a human head,

considered from the frontal perspective, at least one of the curved spring-tempered wires is disposed above the human ear and at least one of the curved spring-tempered wires is disposed underneath the human ear, both on the right and on the left side of the head,

and/or

the plane of symmetry of the external head apparatus is substantially identical to the plane of symmetry passing centrally through the human head.

Herein the term frontal perspective means that the human head is viewed from the front in natural position, i.e. the eyes are directed forward toward the observer and the eyes are positioned above the mouth. The term “substantially identical” means that the planes of symmetry coincide (under the assumption of an ideally symmetric head), aside from small deviations. In other words, this means that the symmetry of the human head is not broken by placing the external head apparatus on it. The term “during fixation of the external head apparatus on a human head” means self-supporting fixation in the intended manner, as was described in the foregoing.

Correspondingly preferred external head apparatuses are particularly advantageous because, despite a large number of conceivable alternative structural possibilities for the design of an external head apparatus, the preferred configurations have proved in practice to be the external head apparatuses in which the highest possible wearing comfort can be combined with the best possible effect on the tone of the masticatory muscles. In addition, the preferred version permits fixation on almost all possible head shapes to be achieved with only a few basic sizes of the external head apparatus and simultaneously permits the desired therapeutic effect to be achieved. Since symmetry of the facial region is usually a major factor for the esthetic perception of human beings, corresponding preferred configurations are usually evaluated in practice as particularly optically attractive, whereby end user acceptance for wearing the external head apparatus can be substantially increased.

Preference is given to an external head apparatus according to the invention (preferably identified as in the foregoing as being preferred) wherein, after fixation of the external head apparatus on a human head, the two separate connecting elements of the external head apparatus are disposed on the back of the head.

Correspondingly preferred external head apparatuses are particularly advantageous, because they are usually evaluated in practice as particularly optically attractive, whereby end user acceptance for wearing the external head apparatus can be substantially increased. Furthermore, the preferred configuration of the external head apparatus permits the most advantageous force transmission by the curved spring-tempered wires. In practice, it has been shown that especially the independent fixability of the external head apparatus is the most advantageous when the connecting elements are situated at the back of the head, and a certain basic spacing from the supports bearing on the muscles is also associated with this.

Preference is given to an external head apparatus according to the invention (preferably identified as in the foregoing as being preferred), wherein the separate connecting elements have, relative to the longest curved spring-tempered wire, a spacing from one another that is larger than 5%, preferably larger than 10%, particularly preferably larger than 15% relative to the length of the corresponding curved spring-tempered wire

and/or

wherein the curved spring-tempered wires extend substantially parallel to one another in the region between the separate connecting elements.

Correspondingly preferred external head apparatuses are particularly advantageous, because they have proved in practice to be particularly durable and load-resistant. To this extent, the advantages described in the foregoing are particularly apparent in corresponding configurations. In particular, the specified minimum spacings alone or in combination with the parallel course of the curved spring-tempered wires between the two separate connecting elements at the back of the head have proved in our own tests as particularly agreeable in terms of wearing properties. Furthermore, preferred configurations with parallel arrangement of the curved spring-tempered wires between the two separate connecting elements can also be made with particularly efficient use of time, especially from kits according to the invention, because the wires extending between the separate connecting elements in this region do not have to be twisted relative to one another.

Preference is given to an external head apparatus according to the invention (preferably identified as in the foregoing as being preferred), wherein the spacing between the separate connecting elements is different relative to both curved spring-tempered wires.

This means that the two separate connecting elements do not lie parallel to one another but instead enclose an angle between one another.

Correspondingly preferred external head apparatuses are particularly advantageous, because a further possibility of influencing the application of pressure of the supports disposed at the ends of the curved spring-tempered wires on the muscles is created by an appropriate arrangement of the connecting elements. For example, a different mechanical response can be generated for two spring-tempered wires of the same length that are identical in terms of their basic mechanical properties when two stiff connecting elements lie close to one another on one wire and far apart from one another, i.e. closer to the supports, for the other wire. Furthermore, an even more individual adaptation of the external head apparatus to the needs of the individual patient is possible.

Preference is given to an external head apparatus according to the invention (preferably identified as in the foregoing as being preferred), wherein the separate connecting elements are set up to permit a change of the spacing between the two curved spring-tempered wires,

and/or

wherein the separate connecting elements are joined to the curved spring-tempered wires in such a way that rotation of the connecting element around the curved spring-tempered wire is possible.

Correspondingly preferred external head apparatuses are particularly advantageous, because they are particularly practical in everyday use and can be independently adapted particularly easily by the patient. Due to the possibility of changing the spacing between the two curved spring-tempered wires by adjusting the connecting elements (e.g. by a change in length of the connecting element, for example by a setscrew, a plug system or a slide mechanism that can be fixed in steps), the patient has the possibility of adapting the wearing properties particularly flexibly to his or her individual needs. In particular, it is therefore possible even for persons with different head sizes and/or shapes to use a common external head apparatus, which can be advantageous especially in hospitals, where the patients are subject to some fluctuation and only a limited inventory of external head apparatuses is available. The possibility of rotating the connecting element around the curved spring-tempered wire not only gives the entire head apparatus a certain flexibility as well as the ability to absorb impacts better, but also permits the entire external head apparatus to be folded flat together and stored in particularly space-saving manner.

Preference is given to an external head apparatus according to the invention (preferably identified as in the foregoing as being preferred), wherein the curved spring-tempered wires are coated with an organic or inorganic protective layer and/or wherein the curved spring-tempered wires are provided with a plastic overlay.

Correspondingly preferred external head apparatuses are particularly advantageous, because the resistance of the curved spring-tempered wires to surface oxidation is increased by the protective layer. Even if this oxidation usually has no negative impacts on the durability of the external head apparatus, it is sometimes interpreted by the end consumers as damage or at least is perceived as a sign of low processing quality, which leads to unnecessary complaints. Prevention of oxidation is therefore particularly advantageous. The coating with a plastic overlay additionally has the advantage that this protects not only the wire but also a purely cosmetic coating that may be present underneath, e.g. a colored lacquer. Furthermore, many patients prefer the feeling of plastic on skin as opposed to the feeling of metal.

Preference is given to an external head apparatus according to the invention (preferably identified as in the foregoing as being preferred), wherein the pressure exerted by the supports on the masticatory muscles during fixation of the external head apparatus on a human head is lower than 60 kPa, preferably lower than 30 kPa, particularly preferably lower than 15 kPa.

Correspondingly preferred external head apparatuses are particularly advantageous, because, according to our own knowledge, the application of an excessive pressure does not act advantageously on the therapeutic benefits but in contrast could possibly be associated with other disadvantages. The basic principle and recipe for success of the external head apparatus according to the invention is the reduction of the tone of the masticatory muscles by continuous gentle loading of the masticatory muscles without the use of high pressures, as would be necessary, for example, for massaging devices (for example, see DE102011016505 A1). To this extent, corresponding preferred external head apparatuses are particularly gentle for the patient.

Preference is given to an external head apparatus according to the invention (preferably identified as in the foregoing as being preferred), wherein the supports disposed at the ends of the curved spring-tempered wires are provided with a macroscopically structured bearing surface, so that the supports generate, along the longest extent of the bearing surface on the human head, an uneven pressure profile during fixation of the external head apparatus on the human head, in particular are provided with a macroscopically structured bearing surface that in axial section through the middle of the support exhibits three or more peaks and two or more valleys

and/or

wherein the supports are provided with electrically operated functional elements, preferably with heating elements and/or vibrating elements and/or sensor elements and/or means for electrical stimulation of muscles.

Correspondingly preferred external head apparatuses are particularly advantageous, because it has been shown in our own tests that, in certain patients who have already suffered from CMD symptoms for a relatively long time and in whom the proportional muscular load of the two large masticatory muscles, the masseter muscle and the temporal muscle, additionally deviates from the norm during the masticatory movement, an increased likelihood of headaches may develop occasionally during use of head apparatuses that are not preferred according to the invention. For these patients, a reduced tendency to headaches and an increased therapeutic effect were found for preferred external head apparatuses in which the supports are provided with a macroscopically structured bearing surface. For example, such a macroscopically structured bearing surface can be generated by attaching two concentric plastic rings with different diameters to the support. For example, four peaks (two for the larger and two for the smaller ring) as well as three valleys between these peaks are obtained hereby in the axial section through the middle of the support. Moreover, or alternatively, it may be advantageous to intensify the therapeutic effect of the external head apparatus by delivering, to the muscle, via the supports provided with corresponding elements, thermal, mechanical or electrical stimuli capable of leading to a further reduction of the tone. Especially from the therapeutic viewpoint, it is additionally desirable to dispose, in the supports, sensor elements via which data about the patient and his or her condition can be collected during use.

The present invention additionally relates to a kit for constructing an external head apparatus according to the invention, comprising
A one or more blanks, comprising
   two spring-tempered wires, wherein the spring-tempered wires are joined to one another via two separate connecting elements,

and
B four or more supports for arrangement at the ends of the spring-tempered wires,

and
C a bending tool for shaping the spring-tempered wires and/or a cutting tool for cutting the spring-tempered wires to length,

as well as optionally
D a work instruction for shaping the spring-tempered wires and/or cutting them to length.

A kit according to the invention permits optimum and efficient marketing of the external head apparatuses according to the invention and offers the greatest benefits for the patients. Especially in complicated CMD cases, prevailing opinion suggests that the best results with the external head apparatus according to the invention can be achieved in the cases in which they are individually matched directly by the treating physician or physiotherapist to the needs of the patient and are regularly adapted to changed circumstances in the course of the therapy. Whereas the construction of an external head apparatus according to the invention does not present particular difficulties to a person skilled in the art, for example of production engineering, in an average workshop, this fabrication capability unfortunately cannot be transferred completely into an average practice, even if a well equipped laboratory is available to it. In our own field tests, it has been encouragingly shown that the final fabrication of an external head apparatus according to the invention can be achieved relatively reliably by physicians, physiotherapists and their technical personnel when suitable blanks, the bending tool and/or cutting tool adapted to the blank and (optionally) a corresponding work instruction are made available to them in the form of a kit. It has been shown that, after the basic processing techniques have been taught and after the needed tool has been provided, the physician or physiotherapist is able to perform at least the bending of the spring-tempered wires and/or the cutting to length of the spring-tempered wires, i.e. those very steps that are particularly important for individual adaptation. For this reason, the kit according to the invention comprises a blank, in which the two spring-tempered wires are already joined by two separate connecting elements and are respectively provided at their ends with supports.

The spring-tempered wires in the blank must be bent into the final shape and/or cut to the correct length corresponding to the needs. This shaping of the blank to correspond to the patient (i.e. the final adaptation to the individual patient) is undertaken by the physician or physiotherapist by using the tool adapted optimally to the wire, the selection of which tool is possible without problems for a person skilled in the art of production engineering (especially if the manufacturer's instructions are available).

For the person skilled in the art, it is obvious that the four or more supports for arrangement at the ends of the spring-tempered wires can also be already disposed at the ends of the spring-tempered wires in a kit according to the invention, especially when it is intended that only bending of the blanks and no cutting to length will be performed by the physician or physiotherapist.

Particularly preferred configurations of the kit according to the invention are derived from the foregoing explanations of preferred external head apparatuses, wherein the blank is respectively adapted correspondingly.

The present invention additionally relates to a a system comprising an external head apparatus according to the invention as well as

at least one additional elastic or reclosable connecting element for joining the two curved spring-tempered wires after fixation of the external head apparatus on a human head,

and/or

a padded or non-padded overlay of fabric or leather for improving the wearing comfort.

Corresponding systems offer even further improved wearing comfort. The additional elastic or reclosable connecting element, for example a rubber band or a band with hook-and-loop fastener for joining the two curved spring-tempered wires is used to fix the external head apparatus particularly securely on the head after fixation of the external head apparatus on a human head. Thereby the use of the external head apparatus is improved even in phases or high load, e.g. during sleep or during sporting activity, since the external head apparatus is no longer able to slip or at least is less easily able to slip. A padded or non-padded overlay of fabric or leather may be pulled over the external head apparatus. It has been shown in interviews that patients regularly prefer the feeling of fabric or leather on the skin as opposed to the feeling of metal or plastic. The wearing comfort is increased particularly strongly when the overlay is padded.

The invention will be explained in more detail hereinafter on the basis of the figures,

FIG. 1 shows an external head apparatus according to the prior art, in which two curved spring-tempered wires (1, 2) are joined to one another with only one single separate connecting element (3), wherein supports (5) are respectively disposed at both ends of the curved spring-tempered wires. FIGS. 2 and 3 show an embodiment of an external head apparatus according to the invention, in which two curved spring-tempered wires (1, 2) are joined to one another according to the invention with two separate connecting elements (3, 4), wherein supports (5) are again respectively disposed at both ends of the curved spring-tempered wires. In this device according to the invention, the curved spring-tempered wires (1, 2) are joined to one another by the separate connecting elements (3, 4) in such a way that the external head apparatus can be fixed on a human head. The principle of fixation of external head apparatuses is known from the literature and disclosed, for example in DE 202011103819 U1. In FIG. 3, the plane of symmetry of the external head apparatus is indicated and marked with α. In FIG. 3, the plane of symmetry α intersects the two curved spring-tempered wires (1, 2) orthogonally, so that the two separate connecting elements (3, 4) are situated on different sides of the plane of symmetry.

The objects mentioned in the foregoing are achieved not only by the external head apparatuses according to the invention but also by the subject matter of the following embodiments, wherein the foregoing explanations of advantages of preferred embodiments as well as the specified definitions are correspondingly applicable.

Embodiment 1 relates to an external head apparatus for reducing the tone of the masticatory muscles in patients with bruxism and/or other craniomandibular dysfunctions (CMD), comprising two curved spring-tempered wires, which are joined to one another by two separate connecting elements, wherein supports are disposed respectively at both ends of the curved spring-tempered wires, wherein the spring-tempered wires have a length of 25 to 48 cm, preferably 28 to 44 cm, wherein the separate connecting elements have a length of 4 to 10 cm, preferably 5 to 8 cm, wherein the spacing of the supports at the two ends of one curved spring-tempered wire from one another is respectively 5 to 11 cm preferably 7 to 9 cm, wherein the two separate connecting elements have a spacing of 3 to 8 cm, preferably 4 to 6 cm from one another, wherein the external head apparatus has a plane of symmetry, which intersects the two curved spring-tempered wires.

Embodiment 2 relates to an external head apparatus according to embodiment 1, wherein the curved spring-tempered wires consist or a stainless steel, and/or wherein the connecting elements consist of a material that is selected from the group consisting of structural and high-performance plastics, preferably is selected from the group consisting of PEEK, PA, POM, PC and PEK, and/or wherein the supports consist of a material that is selected from the group consisting of structural and high-performance plastics, preferably is selected from the group consisting of PEEK, PA, POM, PC and PEK.

Embodiment 3 relates to an external head apparatus according to one of embodiments 1 or 2, wherein the two separate connecting elements, relative to each of the two curved spring-tempered wires, have a spacing of 8 to 22 cm, preferably 10 to 20 cm, from the next closest support.

Embodiment 4 relates to an external head apparatus according to one of embodiments 1 to 3, wherein the curved spring-tempered wires extend substantially parallel to one another in the region between the separate connecting elements.

embodiment 5 relates to an external head apparatus according to one of embodiments 1 to 4, wherein the spacing between the separate connecting elements is different relative to both curved spring-tempered wires.

Embodiment 6 relates to an external head apparatus according to one of embodiments 1 to 5, wherein the separate connecting elements are set up to permit a change of the spacing between the two curved spring-tempered wires and/or wherein the wherein the separate connecting elements are joined to the curved spring-tempered wires in such a way that rotation of the connecting element around the curved spring-tempered wire is possible.

Embodiment 7 relates to an external head apparatus according to one of embodiments 1 to 6, wherein the curved spring-tempered wires are coated with an organic or inorganic protective layer and/or wherein the curved spring-tempered wires are provided with a plastic overlay.

Embodiment 8 relates to an external head apparatus according to one of embodiments 1 to 7, wherein the supports disposed at the ends of the curved spring-tempered wires are provided with a macroscopically structured bearing surface, especially with a macroscopically structured bearing surface that in axial section through the middle of the support exhibits three or more peaks and two or more valleys, and/or wherein the supports are provided with electrically operated functional elements, preferably with heating elements and/or vibrating elements and/or sensor elements and/or means for electrical stimulation of muscles.

Embodiment 9 relates to a kit for constructing an external head apparatus according to one of embodiments 1 to 8, comprising A one or more blanks, comprising two spring-tempered wires, wherein the spring-tempered wires are joined to one another via two separate connecting elements, and B four or more supports for arrangement at the ends of the spring-tempered wires, and C a bending tool for shaping the spring-tempered wires and/or a cutting tool for cutting the spring-tempered wires to length, as well as optionally D a work instruction for shaping the spring-tempered wires and/or cutting them to length.

Embodiment 10 relates to a system comprising an external head apparatus according to one of embodiments 1 to 8 as well as to at least one additional elastic or reclosable connecting element for joining the two curved spring-tempered wires after fixation of the external head apparatus on a human head, and/or to a padded or non-padded overlay of fabric or leather for improving the wearing comfort.

1. An external head apparatus for reducing the tone of the masticatory muscles in patients with bruxism and/or other craniomandibular dysfunctions (CMD), comprising
(i) two curved spring-tempered wires, and
(ii) two separate connecting elements,
wherein supports are disposed respectively at both ends of the curved spring-tempered wires,
wherein the curved spring-tempered wires are joined to one another by the separate connecting elements in such a way that the external head apparatus can be fixed on a human head,
wherein the curved spring-tempered wires joined to one another are shaped in such a way that, during fixation of the external head apparatus on a human head, pressure due to the supports disposed at the ends of the curved spring-tempered wires exerted on trigger points or fasciae of the two large masticatory muscles, the masseter muscle and the temporal muscle, respectively on the right and left side of the human head,
wherein the external head apparatus has a plane of symmetry that intersects the two curved spring-tempered wires, and
wherein the two separate connecting elements are situated on different sides of the plane of symmetry.

2. The external head apparatus according to claim 1, wherein, during fixation of the external head apparatus on a human head,
considered from the frontal perspective, at least one of the curved spring-tempered wires disposed above the human ear and at least one of the curved spring-tempered wires is disposed underneath the human ear, both on the right and on the left side of the head, and
the plane of symmetry of the external head apparatus is substantially identical to the plane of symmetry passing centrally through the human head, and
the two separate connecting elements the external head apparatus are disposed on the back of the head.

3. The external head apparatus according claim 1, wherein the separate connecting elements have, relative to the longest curved spring-tempered wire, a spacing from one another that is larger than 5%, preferably larger than 10%, particularly preferably larger than 15% relative to the length of the corresponding curved spring-tempered wire, and
wherein the curved spring-tempered wires extend substantially parallel to one another in the region between the separate connecting elements.

4. The external head apparatus according to claim 1, wherein the spacing between the separate connecting elements is different relative to both curved spring-tempered wires.

5. The external head apparatus according to claim 1,
wherein the separate connecting elements are set up to permit a change of the spacing between the two curved spring-tempered wires, and
wherein the separate connecting elements are joined to the curved spring-tempered wires in such a way that rotation of the connecting element around the curved spring-tempered wire is possible.

6. The external head apparatus according to claim 1, wherein the curved spring-tempered wires are coated with an organic or inorganic protective layer or wherein the curved spring-tempered wires are provided with a plastic overlay.

7. The external head apparatus according to claim 1, wherein the pressure exerted by the supports on the masticatory muscles during fixation of the external head apparatus on a human head is lower than 60 kPa, preferably lower than 30 kPa, particularly preferably lower than 15 kPa.

8. The external head apparatus according to claim 1,
wherein the supports disposed at the ends of the curved spring-tempered wires are provided with a macroscopically structured bearing surface, so that the supports generate, along the longest extent of the bearing surface on the human head, an uneven pressure profile during fixation of the external head apparatus on the human head, in particular are provided with a macroscopically structured bearing surface that in axial section through the middle of the support exhibits three or more peaks and two or more valleys; and
wherein the supports are provided with electrically operated functional elements, preferably with heating elements or vibrating elements or sensor elements or means for electrical stimulation of muscles.

9. A kit for constructing an external head apparatus according to claim 1, the kit comprising:
one or more blanks, comprising
two spring-tempered wires, wherein the spring-tempered wires are joined to one another via two separate connecting elements, and
four or more supports (5) for arrangement at the ends of the spring-tempered wires; and
a bending tool for shaping the spring-tempered wires or a cutting tool for cutting the spring-tempered wires to length.

10. A system for an external head apparatus, the system comprising:
an external head apparatus according to claim 1,
at least one additional elastic or reclosable connecting element for joining the two curved spring-tempered wires after fixation of the external head apparatus on a human head, and
a padded or non-padded overlay of fabric or leather for improving the wearing comfort.

11. The kit according claim 9, further comprising a work instruction for shaping the spring-tempered wires or cutting spring-tempered wires to length.

12. The external head apparatus according to claim 1, wherein, during fixation of the external head apparatus on a human head,
considered from the frontal perspective, at least one of the curved spring-tempered wires is disposed above the human ear and at least one of the curved spring-tempered wires is disposed underneath the human ear, both on the right and on the left side of the head,
or
the plane of symmetry of the external head apparatus is substantially identical to the plane of symmetry passing centrally through the human head,
and
the two separate connecting elements of the external head apparatus are disposed on the back of the head.

13. The external head apparatus according to claim 1, wherein, during fixation of the external head apparatus on a human head,
considered from the frontal perspective, at least one of the curved spring-tempered wires is disposed above the human ear and at least one of the curved spring-tempered wires is disposed underneath the human ear, both on the right and on the left side of the head, and
the plane of symmetry of the external head apparatus is substantially identical to the plane of symmetry passing centrally through the human head,
or
the two separate connecting elements of the external head apparatus are disposed on the back of the head.

14. The external head apparatus according claim 1, wherein the separate connecting elements have, relative to the longest curved spring-tempered wire, a spacing from one another that is larger than 5%, preferably larger than 10%, particularly preferably larger than 15% relative to the length of the corresponding curved spring-tempered wire, or
wherein the curved spring-tempered wires extend substantially parallel to one another in the region between the separate connecting elements.
